(19) United States
(12) Patent Application Publication
(10) Pub. No.: US 2004/0127467 A1
(43) Pub. Date: Jul. 1, 2004

(54) Synthesis of pancratistatin prodrugs

(76) Inventors: George R Pettit, Paradise Valley, AZ (US); Brian Orr, Del Mar, CA (US); Sylvie Ducki, Salford (GB)

Correspondence Address: Susan Stone Rosenfield, Fennemore Craig, Suite 2600, 3003 North Central, Phoenix, AZ, 85012-2913, US

(21) Appl. No.: 10/475,061

(22) PCT Filed: Apr. 17, 2002
(86) PCT No.: PCT/US02/11964

(51) Int. Cl. A61K 31/665

(52) U.S. Cl. 514/99

(57) ABSTRACT

A new and efficient synthesis of the (+)-pancratistatin phosphate prodrug 2a has been accomplished. Selective protection (tetraacetate 4) of (+)-pancratistatin (1a) was followed by phosphorylation (to 5) with dibenzyl chlorophosphite (prepared in situ from dibenzyl phosphite). Cleavage of the acetate (with sodium methoxide) and benzyl (by hydrogenolysis) protecting groups followed by concomitant reaction with two equivalents of sodium methoxide afforded good yield of disodium (+)-pancratistatin phosphate (2a). Further increases in yields of the prodrug (2a) were realized by avoiding heat in the final purification steps. Fourteen (2b-o) additional metal and ammonium derived phosphate prodrugs were also synthesized.

This application claims the benefit of U.S. provisional application filed on Apr. 19, 2001.

This research was funded in part by Outstanding Investigator Grant CA 44344-01-11 awarded by the National Cancer Institute, DHHS. The United States Government may have certain rights to this invention.

### INTRODUCTION

This invention relates generally to the discovery of a new and efficient synthesis of the (+)-pancratistatin phosphate prodrug and fourteen (14) metal and ammonium cation derivatives thereof. The pancratistatin phosphate prodrug denominated 2a in the enclosed Scheme was found to have exceptional solubility in water and therefore readily administerable in the treatment of human cancer.

### BACKGROUND OF THE INVENTION

The anticancer phenanthridone (+)-pancratistatin (1a) was isolated from the bulbs of the Hawaiian Hymenocallis (formally Pancratium) littoralis (Pettit et al., 1984a, Journal of the Chemical Society, Chemical Communications, 1693; Pettit et al., 1984b, Journal of Natural Products, 47, 1018). The structure was determined by NMR spectroscopy and confirmed by x-ray crystallographic analysis of its 7-methoxy derivative (1b). Subsequently, pancratistatin (1a) was found to exhibit strong in vitro cancer cell growth inhibitory activities including against the U.S. National Cancer Institute (NCI) panel of cancer cell lines (Pettit et al., 1986, Journal of Natural Products, 49, 995; 1993 Journal of Natural Products, 56, 1682) and a series of in vivo experimental cancer systems. A related area of promise involves its activity against a range of RNA viruses (Gabrielsen et al., 1992, Journal of Natural Products, 55, 1569). Due to the relatively low natural abundance (˜0.039% of dry bulb) and its increasing potential as a clinically useful antitumor agent, pancratistatin has become an important target for total synthesis. The first total synthesis of (±)-pancratistatin was reported in 1989 (Danishefsky and Yon Lee, 1989, Journal of the American Chemical Society, 111, 4829). Six stereospecific syntheses of natural (+)-pancratistatin (1a) were subsequently completed (Tian et al., 1995, Journal of the American Chemical Society, 117, 3643; Trost and Pulley, 1995, Journal of the American Chemical Society, 117, 10143; Hudlicky et al, 1996, Journal of the American Chemical Society, 118, 10752; Doyle et al., 1997, Tetrahedron, 53, 11153; Magnus and Sebhat, 1998, Tetrahedron, 54, 15509; Pettit et al, 2000, Journal of Organic Chemistry, in preparation). Meanwhile, a number of partial syntheses have been described (Lopes et al., 1992, Tetrahedron Letters, 33, 6775; Angle and Louie, 1993, Tetrahedron Letters, 34, 4751; Grubb et al., 1999, Tetrahedron Letters, 40, 2691).

The clinical development of pancratistatin (1a) was earlier hampered due to its poor solubility in water (<53 μg/ml). This problem was addressed (Pettit et al., 1995a, Anti-Cancer Drug Design, 10, 243) by developing a synthesis of the phosphate prodrug (2a) which displayed greatly improved solubility characteristics in water and was found to exhibit equal activity against the murine P388 lymphocytic leukemia cell. Presumably, attachment of a phosphate is also beneficial for in vivo systems since human non-specific phosphatases will hydrolyze the phosphate prodrug following administration to release (+)-pancratistatin (1a). Since the relatively unstable phosphorylating agent, dibenzyl-(N,N-diisopropylamido)-phosphine, we employed in our initial synthesis of phosphate (2a), was less than desirable and yields for the phosphorylation reaction itself proved variable (0-91% yields of (5) with no recovery of valuable starting material), we thus investigated less astringent approaches. Herein, an alternate simpler synthesis of pancratistatin prodrug (2a) will be described. The new synthesis will be used to provide the pure drug (2a) in quantity for preclinical development. In addition, a series of phosphate metal and ammonium cation salt derivatives (2b-o) was prepared in order to evaluate effects on human cancer cell growth 30 and solubility properties (in water).

These and still further objects as shall hereinafter appear are readily fulfilled by the present invention in a remarkably unexpected manner as will be readily discerned from the following detailed description of an exemplary embodiment thereof.

### BRIEF DESCRIPTION OF DRAWINGS

FIG. 1 is a schematic showing of the step-by-step synthesis of the present invention.

### DETAILED DESCRIPTION OF PREFERRED EMBODIMENT

(+)-Pancratistatin (1a) was isolated from Hymenocallis littoralis as previously described (Pettit et al., 1995b, Journal of Natural Products, 58, 37). Acetic anhydride, pyridine, dibenzyl phosphite, N-ethyldiisopropylamine and 10% palladium over carbon catalyst were purchased from Lancaster Synthesis, Inc. Carbon tetrachloride, 4-dimethylaminopyridine, anhydrous magnesium sulfate (MgSO₄), potassium dihydrogenphosphate, phosphomolybdic acid, and zinc acetate dihydrate were from Sigma-Aldrich Chemical Co. Sodium methoxide, lithium hydroxide monohydrate, piperazine (anhydrous) and morpholine were from Acros Organics. Calcium acetate and manganese acetate were purchased from Fisher Scientific Co. and magnesium acetate quinine, quinidine and imidazole from J. T. Baker Chem. Rubidium carbonate and cesium carbonate were supplied by Alfa Products, Inc. and potassium acetate from Mallinckrodt. All solvents were redistilled prior to use and dried when necessary. Solvent extracts of aqueous solutions were dried over magnesium sulfate. The 1.0 M solutions of the metal bases were prepared in distilled water and the 1.0 M solutions of the amines in dry methanol.

All reactions were carried out under an atmosphere of nitrogen unless otherwise specified and their progress was ascertained by thin-layer chromatography using Analtech silica gel GHLF Uniplates (visualized under long- and short-waves UV) and developed in an ethanolic solution of phosphomolybdic acid reagent. Column chromatography was performed with silica gel 60 (230-400 mesh) from E. Merck. The Sephadex® G-10 was washed (prior to use) with 1 N sodium hydroxide, water, 1 N acetic acid and finally water until neutral pH.

All melting points were determined with an Electrothermal digital melting point apparatus model IA9200 and are uncorrected. Optical rotation values were recorded using a Perkin Elmer 241 polarimeter. The IR spectra were from a Nicolet FTIR model MX-1 instrument. EIMS spectra were obtained with a MAT 312 mass spectrometer; high- and low-resolution FAB spectra were obtained with a Kratos MS-50 mass spectrometer (Midwest Center for Mass Spectrometry, University of Nebraska, Lincoln, Nebr.) using a glycerol-triglycerol matrix. The ¹H-, ¹³C- and ³¹P-NMR spectra were recorded with Varian Gemini 300, 400 or 500 MHz instruments. Elemental analyses were determined by Galbraith Laboratories, Inc. (Knoxville, Tenn.).

1,2,3,4-O-Tetraacetoxy-pancratistatin (4)—The following procedure represents a useful improvement over our earlier method (Pettit et al., 1995a, Anti-Cancer Drug Design, 10, 243). To a stirring solution of (+)-pancratistatin (1a, 82% pure, 8.6 g, 21.7 mmol) in pyridine (50 ml) were added acetic anhydride (45 ml, 0.48 mol) and 4-dimethylaminopyridine (200 mg, 1.6 mmol) at room temperature. After stirring for 16 hours, ice (100 ml) was added to the mixture and stirring was continued for a further 1 hour. The resultant mixture was extracted with dichloromethane (2×100 ml) and the combined organic extracts were dried, filtered and evaporated in vacuo.

Pyridine (80 ml) and water (40 ml) were added to the brown oily residue (14.3 g) and the mixture was heated under reflux for 1 hour. Ice (50 ml) was added and the cooled mixture was extracted with dichloromethane (2×100 ml). The combined organic extract was dried, filtered and solvent evaporated in vacuo. The resulting residue was purified by column chromatography on silica gel eluting with 1.5% methanol in dichloromethane, affording a white amorphous powder, which was recrystallized from ethanol to give the title compound (4, 7.1 g, 66%) as colorless needles; m.p. 240° C. [m.p. lit. (Pettit et al., 1995a, Anti-Cancer Dug Design, 10, 243) 243-246° C.]; [α]_{D}²⁷+31.5° (c 1.04, DCM) {[α]_{D}³⁵ lit. (Pettit et al, 1995a, Anti-Cancer Drug Design, 10, 243) +30.4° (c 0.72, DCM)}. The resulting IR, ¹H-NMR and ¹³C-NMR spectra were as previously reported.

1,2,3,4-O-Tetraacetoxy-7-O-dibenzyloxyphosphoryl-pancratistatin (5)—To a solution of 1,2,3,4-O-tetraacetoxy-pancratistatin (4, 2.60 g, 5.27 mmol) in acetonitrile (30 ml), cooled to −30° C. (ethylene glycol/dry ice), were added carbon tetrachloride (2.6 ml, 26.9 mmol), N-ethyldiisopropylamine (2.0 ml, 11.6 mmol) and 4-dimethylaminopyridine (71 mg, 0.58 mmol). Following the slow dropwise addition of dibenzyl phosphite (1.80 ml, 8.15 mmol), the mixture was stirred for 3 hours. The reaction was terminated by addition of an aqueous solution of potassium dihydrogenphosphate (0.5 M, 50 ml) and stirred at room temperature for 30 minutes. The resultant mixture was extracted with dichloromethane (2×50 ml) and the combined organic extract was dried, filtered and solvent evaporated (in vacuo). The resulting yellow oil was purified, at low temperature (4° C.), by column chromatography on silica gel and eluting with 0.8% methanol in dichloromethane. That procedure afforded recovered starting material, 1,2,3,4-O-tetraacetoxy-pancratistatin (4, 0.30 g, 11%), closely followed by its dibenzyl phosphate derivative (5, 3.44 g, 87%) as a colorless crystalline solid; m.p. 119° C. [m.p. lit. (Pettit et al., 1995a, Anti-Cancer Drug Design, 10, 243) 119-121° C.]; [α]_{D}²⁷+59.3° (c 1.33, DCM) {[α]_{D}³³ lit. (Pettit et al., 1995a, Anti-Cancer Drug Design, 10, 243) +69.1° (c 0.89, DCM)}; vₘₐₓ (KBr disc) 2910w, 1755s, 1674s, 1485s, 1373s, 1221s, 1037b, 871w, 738w, 493w cm⁻¹; δ_{H}/ppm (300 MHz, CDCl₃) 2.04 (3H, s, OAc), 2.06 (3H, s, OAc), 2.07 (3H, s, OAc), 2.16 (3H, s, OAc), 3.40 (1H, dd, J3, 13 Hz, H-10b), 4.26 (1H, dd, J11, 13 Hz, H-4a), 5.13 (1H, dd, J3, 11 Hz, H-4), 5.20 (1H, t, J3 Hz, H-2), 5.23 (1H, dd, J7, 12 Hz, CH_{A}H_{B}Ph), 5.26 (1H, dd, J7, 12 Hz, CH_{A}H_{B}Ph), 5.31 (1H, dd, J7, 12 Hz, CH_{C}H_{D}Ph), 5.41 (1H, dd, J7, 12 Hz, CH_{C}H_{D}Ph), 5.43 (1H, t, J3 Hz, H-3), 5.54 (1H, t, J3 Hz, H-1), 5.92 (1H, d, J1 Hz, OCH_{A}H_{B}O), 5.95 (1H, d, J1 Hz, OCH_{A}H_{B}O), 6.10 (1H, bs, 5-NH), 6.43 (1H, s, H-10), 7.30-7.42 (10H, bm, 2×Ph); δ_{C}/ppm (125 MHz, CDCl₃) 170.1 (C═O at C-1), 169.7 (C═O at C-3), 169.0 (C═O at C-4), 168.2 (C═O at C-2), 162.6 (C-6), 152.4 (C-9), 139.3 (d, J_{PC} 4 Hz, C-8), 136.0 (d, J_{PC} 8 Hz, C of Ph), 135.9 (d, J_{PC} 8 Hz, C of Ph), 134.1 (d, J_{PC} 7 Hz, C-7), 133.0 (C-10a), 128.42 (CH of Ph), 128.37 (CH of Ph), 128.3 (CH of Ph), 127.9 (CH of Ph), 127.7 (CH of Ph), 117.0 (C-6a), 102.7 (OCH₂O), 101.5 (C-10), 71.6 (C-4), 70.1 (d, J_{PC} 5 Hz, CH₂Ph), 70.0 (d, J_{PC} 6 Hz, CH₂Ph), 67.6 (C-2), 66.7 (C-3), 66.4 (C-1), 47.6 (C-4a), 40.3 (C-10b), 20.76 (CH₃ at C-2), 20.73 (CH₃ at C-3), 20.67 (CH₃ at C-4), 20.5 (CH₃ at C-1); δₚ/ppm (200 MHz, CDCl₃, referenced to 85% H₃PO₄) −6.61 (s, ¹H decoupled); m/z (EI) 493 [M-P(O)(OBn)₂, 10%], 271 (35), 91 (40), 61 (40), 44 (100).

Disodium 7-O-phosphoryl-pancratistatin (2a)—To a solution of dibenzyl pancratistatin phosphate (5a, 2.00 g, 2.66 mmol) in methanol (50 ml) was added sodium methoxide (87 mg, 1.61 mol). The mixture was stirred at room temperature for 2 hours and then water (50 ml) was added. The aqueous mixture was extracted with dichloromethane (5×50 ml) and the combined organic extract was dried, filtered and evaporated in vacuo (NO HEAT) to give the crude deacetylated derivative as a white powder (1.8 g). The residue was promptly dissolved in ethanol (50 ml) and 10% Pd/C catalyst (201 mg) was added. The mixture was stirred under 1 atm. of hydrogen for 2 hours and filtered through fluted filter paper. The catalyst was washed thoroughly with methanol (50 ml) and the filtrate was evaporated in vacuo (NO HEAT) to give the crude debenzylated phosphoric acid (6) as a white powder (1.3 g); m.p. 195° C. dec.; δ_{H}/ppm (300 MHz, D₂O) 6.70 (1H, bs, H-10), 5.96 (1H, s, OCH_{A}H_{B}O), 5.93 (1H, s, OCH_{A}H_{B}O), 4.29 (1H, bs, H-1), 4.03 (1H, bs, H-2), 3.87 (1H, bs, H-3), 3.74 (1H, m, H-4), 3.67 (1H, t, J11 Hz, H-4a), 3.00 (1H, d, J12 Hz, H-10b); m/z (FAB) 406 [(M+H)⁺, 20%], 405 [M⁺, 25], 154 (100); HRFAB 405.0467 calculated for C₁₄H₁₆NO₁₁P 405.0461.

The phosphoric acid (6) was immediately redissolved in methanol (50 ml) and sodium methoxide (361 mg, 6.73 mmol) was added. The mixture was stirred overnight and then concentrated in vacuo (NO HEAT) to give a white paste (1.6 g). The residue was purified on a Sephadex® G-10 column eluting with water. The fluorescent fractions were combined and freeze dried to give di-sodium pancratistatin prodrug (2a) as a fluffy powder (1.1 g, 92%); m.p. 206° C. (dec); [α]_{D}²⁸+78.4° (c 1.02, H₂O); vₘₐₓ (KBr disc) 3500-3000b, 2360w, 1670bs, 1480s, 1350w, 1090s, 980m, 720m cm⁻¹; δ_{H}/ppm (400 MHz, D₂O) 6.59 (1H, s, H-10), 5.96 (1H, s, OCH_{A}H_{B}O), 5.87 (1H, s, OCH_{A}H_{B}O), 4.39 (1H, s, H-1), 4.11 (1H, bs, H-2), 3.93 (1H, bs, H-3), 3.81 (1H, bd, J9 Hz, H-4), 3.73 (1H, bt, J12 Hz, H-4a), 3.00 (1H, bd, J12 Hz, H-10b); δ_{C}/ppm (100 MHz, D₂O) 171.2 (C═O), 152.3 (C-9), 139.1 (C-8), 137.3 (C-7), 135.3 (C-10a), 117.6 (C-6a), 102.4 (O—CH₂—O), 101.0 (C-10), 72.7 (C-3), 70.7 (C-2), 70.3 (C-4), 68.8 (C-1), 49.1 (C-4a), 40.7 (C-10b); δₚ/ppm (162 MHz, D₂O, referenced to 85% H₃PO₄) 0.90 (s, ¹H decoupled); m/z (FAB) 449 (M⁺, 20%), 427 [(M-Na)⁺, 20), 154 (100).

General procedure for synthesis of the pancratistatin prodrugs (2b-o): To an aqueous methanol solution of the phosphoric acid derivative of pancratistatin (6, 50 mg in 1 ml water or methanol) was added a 1.0 M solution (250 μl) of the appropriate metal (carbonate or acetate) salt or amine free base. The solution became cloudy and the mixture was stirred for 6 hours. The mixture was freeze dried (or evaporated) to afford the desired prodrug salt (2b-o). The salts were further purified by trituration with wet methanol and/or diethylether to remove un-reacted starting materials.

Dilithium pancratistatin 7-O-phosphate (2b): Yellowish powder (61 mg); m.p. 240° C. dec.: δ_{H}/ppm (300 MHz, D₂O) 6.62 (1H, bs, H-10), 600 (1H, s, OCH_{A}H_{B}O), 5.90 (1H, s, OCH_{A}H_{B}O), 4.44 (1H, bs, H-1), 4.15 (1H, bs, H-2), 3.97 (1H, bs, H-3), 3.80 (2H, m, H-4, H-4a), 3.07 (1H, d, J12 Hz, H-10b).

Dipotassium pancratistatin 7-O-phosphate (2c): Colorless powder (66 mg); m.p. 280° C. dec.; δ_{H}/ppm (300 MHz, D₂O) 6.68 (1H, bs, H-10), 6.00 (1H, s, OCH_{A}H_{B}O), 5.95 (1H, s, OCH_{A}H_{B}O), 4.45 (1H, bs, H-1), 4.16 (1H, bs, H-2), 3.97 (1H, bs, H-3), 3.89 (1H, m, H-4), 3.78 (1H, m, H-4a), 3.13 (1H, d, J12 Hz, H-10b).

Dirubidium pancratistatin 7-O-phosphate (2d): Colorless powder (0.11 g); m.p. 200° C. (dec.); δ_{H}/ppm (300 MHz, D₂O) 6.63 (1H, bs, H-10), 6.00 (1H, s, OCH_{A}H_{B}O), 5.90 (1H, s, OCH_{A}H_{B}O), 4.45 (1H, bs, H-1), 4.16 (1H, bs, H-2), 3.98 (1H, bs, H-3), 3.80 (2H, m, H-4, H-4a), 3.10 (1H, d, J12 Hz, H-10b); m/z (FAB) 573.9 [(M+H)⁺, 5%], 298.9 (35), 215.1 (25), 135.1 (100); HRFAB 573.8598 calculated for C₁₄H₁₅NO₁₁PRb₂ 573.8619.

Dicesium pancratistatin 7-O-phosphate (2e): Yellow oil (0.22 g); δ_{H}/ppm (300 MHz, D₂O) 6.63 (1H, bs, H-10), 6.01 (1H, s, OCH_{A}H_{B}O), 5.90 (1H, s, OCH_{A}H_{B}O), 4.46 (1H, bs, H-1), 4.17 (1H, bs, H-2), 3.98 (1H, bs, H-3), 3.85 (1H, m, H-4), 3.79 (1H, m, H-4a), 3.08 (1H, d, J12 Hz, H-10b); m/z (FAB) 669.9 [(M+H)⁺, 50%], 225.0 (100); HRFAB 669.8468 calculated for C₁₄H₁₅NO₁₁PCs₂ 669.8491.

Magnesium pancratistatin 7-O-phosphate (2j): Colorless powder (64 mg); m.p. 220° C. dec.; δ_{H}/ppm (300 MHz, D₂O) 6.66 (1H, bs, H-10), 6.01 (1H, s, OCH_{A}H_{B}O), 5.94 (1H, s, OCH_{A}H_{B}O), 4.44 (1H, bs, H-1), 4.16 (1H, bs, H-2), 3.98 (1H, bs, H-3), 3.80 (2H, m, H-4, H-4a), 3.07 (1H, d, J12 Hz, H-10b).

Calcium pancratistatin-7-O-phosphate (2g): Colorless powder (77 mg); m.p. 230° C. (dec.); δ_{H}/ppm (300 MHz, D₂O) 6.64 (1H, bs, H-10), 5.99 (1H, s, OCH_{A}H_{B}O), 5.96 (1H, s, OCH_{A}H_{B}O), 4.41 (1H, bs, H-1), 4.14 (1H, bs, H-2), 3.97 (1H, bs, H-3), 3.76 (2H, m, H-4, H-4a), 3.41 (1H, d, J11 Hz, H-10b).

Zinc pancratistatin 7-O-phosphate (2h): Colorless crystalline powder (76 mg); m.p. 190° C. (dec.); δ_{H}/ppm (300 MHz, D₂O) 6.69 (1H, bs, H-10), 5.96 (2H, bs, OCH₂O), 4.44 (1H, bs, H-1), 4.15 (1H, bs, H-2), 3.98 (1H, bs, H-3), 3.80 (2H, m, H-4, H-4a), 3.12 (1H, d, J12 Hz, H-10b).

Manganese pancratistatin 7-O-phosphate (2i): Colorless powder (88 mg); m.p. 240° C. (dec.); δ_{H}/ppm (300 MHz, D₂O) 6.65 (1H, bs, H-10), 6.00 (1H, s, OCH_{A}H_{B}O), 5.95 (1H, s, OCH_{A}H_{B}O), 4.44 (1H, bs, H-1), 4.15 (1H, bs, H-2), 3.99 (1H, bs, H-3), 3.79 (2H, m, H-4, H-4a), 3.08 (1H, d, J12 Hz, H-10b).

Piperazine pancratistatin 7-O-phosphate (2j): Colorless powder (78 mg); m.p. 200° C. (dec.); δ_{H}/ppm (300 MHz, D₂O) 6.62 (1H, bs, H-10), 5.99 (1H, s, OCH_{A}H_{B}O), 5.88 (1H, s, OCH_{A}H_{B}O), 4.44 (1H, bs, H-1), 4.15 (1H, bs, H-2), 3.96 (1H, bs, H-3), 3.84 (1H, m, H-₄), 3.77 (1H, m, H-4a), 3.10 (1H, d, J12 Hz, H-10b), 2.99-3.01 (8H, m, 4×CH₂ piperazine); m/z (FAB) 492.2 [(M+H)⁺, 30%), 460.1 (85), 154.1 (100); HRFAB 492.1373 calculated for C₁₈H₂₇N₃O₁₁P 492.1383.

Morpholine pancratistatin 7-O-phosphate (2k): Colorless powder (71 mg); m.p. 180° C. (dec.); δ_{H}/ppm (300 MHz, D₂O) 6.63 (1H, bs, H-10), 5.99 (1H, s, OCH_{A}H_{B}O), 5.89 (1H, s, OCH_{A}H_{B}O), 4.44 (1H, bs, H-1), 4.15 (1H, bs, H-2), 3.96 (1H, bs, H-3), 3.78-3.82 (6H, m, H-4, H-4a, 2×CH₂N morpholine), 3.10-3.14 (5H, m, H-10b, 2×CH₂O morpholine); m/z (AB) 493.2 [(M+H)⁺, 15%], 406.1 (30), 154.1 (00); HRFAB 493.1215 calculated for C₁₈H₂₆N₂P₁₂P 493.1222.

Pyridine pancratistatin 7-O-phosphate (2l): Colorless powder (63 mg); m.p. 270° C. (dec.); δ_{H}/ppm (300 MHz, D₂O) 8.66 (2H, d, J7 Hz, H-2 and H-6 pyridine), 8.49 (1H, t, J7 Hz, H-4 pyridine), 7.97 (2H, t, J7 Hz, H-3 and H-5 pyridine), 6.45 (1H, bs, H-10), 5.95 (2H, s, OCH₂O), 4.41 (1H, bs, H-1), 4.15 (1H, bs, H-2), 3.97 (1H, bs, H-3), 3.89 (1H, m, H-4), 3.84 (1H, m, H-4a), 3.10 (1H, d, J12 Hz, H-10b).

Imidazole pancratistatin 7-O-phosphate (2m): Colorless powder (71 mg); m.p. 250° C. (dec.); δ_{H}/ppm (300 MHz, D₂O) 8.21 (1H, s, H-2 imidazole), 7.21 (2H, bs, H-4 and H-5 imidazole), 6.65 (1H, bs, H-10), 6.00 (1H, s, OCH_{A}H_{B}O), 5.95 (1H, s, OCH_{A}H_{B}O), 4.44 (1H, bs, H-1), 4.15 (1H, bs, H-2), 3.97 (1H, bs, H-3), 3.85 (1H, m, H-4), 3.75 (1H, m, H-4a), 3.38 (1H, d, J12 Hz, H-10b).

Quinine pancratistatin 7-O-phosphate (2n): White fluffy powder (0.11 g); m.p. 173-175° C.; δ_{H}/ppm (300 MHz, D₂O) 8.59 (1H, d, J4.5 Hz, H-2′ quinine), 7.86 (1H, d, J9 Hz, H-8′ quinine), 7.57 (1H, d, J4.5 Hz, H-3′ quinine), 7.36 (1H, dd, J2, 9 Hz, H-5′ quinine), 7.23 (1H, m, H-7′ quinine), 6.34 (1H, s, H-10), 5.91 (1H, s, OCH_{A}H_{B}O), 5.87 (1H, s, OCH_{A}H_{B}O), 5.66 (1H, m, H-10 quinine), 5.59 (1H, m, H-9 quinine), 4.98 (1H, d, J17 Hz, H-11a quinine), 4.93 (1H, d, J11 Hz, H-11b quinine), 4.35 (1H, bs, H-1), 4.12 (1H, bs, H-2), 3.95 (1H, bs, H-3), 3.83-3.87 (5H, m, OMe quinine, H-4, H-4a), 3.80 (1H, m, H-6a quinine), 3.22 (2H, m, H-8 quinine, H-2a quinine), 2.93 (1H, d, J12 Hz, H-10b), 2.77 (2H, m, H-2b quinine, H-6b quinine), 2.04 (1H, m, H-3 quinine), 1.93 (2H, m, H-5a quinine, H-7a quinine), 1.86 (1H, m, H-4 quinine), 1.62 (1H, m, H-5b quinine), 1.27 (1H, m, H-7b quinine); m/z (FAB) 730.2 [(M+H)⁺, 60%], 325.2 (100); HRFAB 730.2372 calculated for C₃₄H₄₁N₃O₁₃P 730.2377.

Quinidine pancratistatin 7-O-phosphate (2o): Colorless powder (0.14 g); m.p. 183-185° C.; δ_{H}/ppm (300 MHz, D₂O) 8.62 (1H, d, J4.5 Hz, H-2′ quinidine), 7.88 (1H, d, J9 Hz, H-8′ quinidine), 7.61 (1H, d, J4.5 Hz, H-3′ quinidine), 7.41 (1H, dd, J2, 9 Hz, H-5′ quinidine), 7.25 (1H, m, H-7′ quinidine), 6.34 (1H, s, H-10), 5.89 (2H, s, OCH₂O), 5.66 (1H, m, H-10 quinidine), 5.59 (1H, m, H-9 quinidine), 5.09 (1H, d, J17 Hz, H-11a quinidine), 4.95 (1H, d, J11 Hz, H-11b quinidine), 4.35 (1H, bs, H-1), 4.14 (1H, bs, H-2), 3.95 (1H, bs, H-3), 3.85-3.89 (5H, m, OMe quinidine, H-4, H-4a), 3.79 (1H, m, H-6a quinidine), 3.23 (2H, m, H-8 and H-2a quinidine), 2.93 (1H, d, J12 Hz, H-10b), 2.65 (2H, m, H-2b and H-6b quinidine), 2.04 (1H, m, H-3 quinidine), 1.88 (2H, m, H-5a and H-7a quinidine), 1.86 (1H, m, H-4 quinidine), 1.69 (1H, m, H-5b quinidine), 1.44 (1H, m, H-7b quinidine); m/z (FAB)730 [(M+H)⁺, 5%], 325 (65), 154 (100); HRFAB 730.2384 calculated for C₃₄H₄₁N₃O₁₃P 730.2377.

Results and Discussion

From the outset, a more direct approach to the protected phosphate intermediate (5) was sought. In this respect, utilization of dibenzyl phosphite techniques (Silverberg et al., 1996, Tetrahedron Letters, 37, 771) by our group (Pettit et al., 1998, Anti-Cancer Drug Design, 13, 981) has proven to be an invaluable alternative to the alkylamidophosphine method (Perrich and Johns, 1988. Synthesis, 142). Due to its poor solubility in organic solvents and the presence of four secondary alcohol groups in the C-ring, upon applying our carefully investigated dibenzyl phosphite methods, direct phosphorylation of (+)-pancratistatin (1a) gave a very complex mixture of products as well as some unreacted starting material. Our attention was therefore focused on selective acetylation of the pancratistatin C-ring hydroxyl groups to give a more soluble and partially protected starting material for the phosphorylation. That objective was easily realized when pancratistatin (1a) was simply allowed to react with acetic anhydride and 4-dimethylaminopyridine in pyridine to give the 1,2,3,4,7-O-pentaacetoxy derivative (3) and then immediately converted to the desired 1,2,3,4-O-tetraacetate (4) by heating in refluxing water-pyridine (66% yield). Reaction of the latter with dibenzyl phosphite in the presence of carbon tetrachloride, N-ethyldiisopropylamine and 4-dimethylaminopyridine afforded the heat sensitive pancratistatin 7-O-dibenzylphosphate (5) in 87% yield. Prolonged contact of (5) with heat and/or silica gel gave phosphate cleavage back to (4). The benzyl phosphate (5) was next deacetylated in the presence of sodium methoxide. Catalytic hydrogenation was used to cleave the benzyl ester groups, affording the corresponding phosphoric acid derivative (6). The acid was immediately treated with two equivalents of sodium methoxide to yield (92%) the disodium phosphate prodrug (2a).

Once this more practical synthesis of (+)-pancratistatin prodrug (2a) was in hand, we proceeded to synthesize and evaluate a variety of cation derivatives of the parent phosphate. By treating the prodrug phosphoric acid precursor (6) with the appropriate base, the required salts (2b-o) were formed. By replacing the sodium cations in prodrug (2a) with different cations, we were able to evaluate water solubility characteristics (mg/ml) as shown in Table II. Some of the ammonium cations were also explored with the goal of obtaining a stable, water-soluble drug with the ability to reverse multidrug resistance through interference with the p-glycoprotein mechanism (Sato et al., 1995, Cancer Chemotherapy and Pharmacology, 35, 271).

All of the synthetic products were evaluated against the murine P388 lymphocytic leukemia cell line and a minipanel of human cancer cell lines. The results are summarized in Table I. Most of the prodrug derivatives exhibited activities analogous to that of (+)-pancratistatin (1a) and the sodium prodrug (2a). Although the manganese (2i) and morpholine (2k) derivatives showed a 10- to 20-fold increase in activity, their poor water solubility made questionable their use for further preclinical development as potential prodrugs. Consequentially, the sodium derivative (2a) continues to be the preferred choice owing to its high activity, adequate water solubility, and the efficient synthesis described herein.

From the foregoing it becomes readily apparent new and useful antineoplastic preparations have been herein described and illustrated which fulfill all of the aforestated objectives. It is of course understood that such modifications, alterations and adaptations as will readily occur to the artisan confronted with this disclosure are intended within the scope of the invention.

1. A method of synthesizing phosphate prodrug comprising selectively protecting (+)-pancratistatin with a tetra acetate and thereafter phospharlating said protected pancratistatin with dibenzyl chloro phosphite; clearing the acetate and benzyl protecting groups with sodium methoxide while reacting with two equivalents of sodium methoxide to yield disodium (+)-pancratistatin phosphate.

2. The method of claim 1 in which the acetate and benzyl protecting groups are cleaned at room temperature.

3. The method of claim 1 in which is the sodium methoxide is replaced by a methoxide having and anion selected from the group consisting of lithium, potassium, risbridium, cesium, magnesium, calcium, zinc, manganese, piperazine, morpholine, pyridine, imidazoles, quinine, and quinidine.

4. The method of claim 3 in which the acetate and benzyl protective groups are cleaned at room temperature.

5. An improved method of synthesizing pancratistatin prodrug comprising, selecting pancratistatin as a starting material and obtaining a protected phosphate intermediate by means of utilizing dibenzyl phosphite techniques.

6. A method according to claim 5 containing the additional steps of: selectively acetylating the C ring hydroxyl groups of said pancratistatin to obtain a pentaacetoxy derivative.

7. A method according to claim 6 containing the additional step of converting said pentaacetoxy derivative to a tetraacetate derivative.

8. A method according to claim 7 containing the additional step of reacting said tetraacetate with said dibenzyl phosphite in the presence of carbon tetrachloride to obtain a dibenzylphosphate derivative.

9. A method according to claim 8 wherein said dibenzyl phosphate was deacetylated in the presence of sodium methoxide.

10. A method according to claim 9 containing the additional step of catalytic hydrogenation of said deacetylated dibenzyl phosphate.

11. A method according to claim 9 containing the additional step of treating said phosphoric acid derivative with sodium methoxide to obtain a disodium phosphate pancratistatin prodrug.

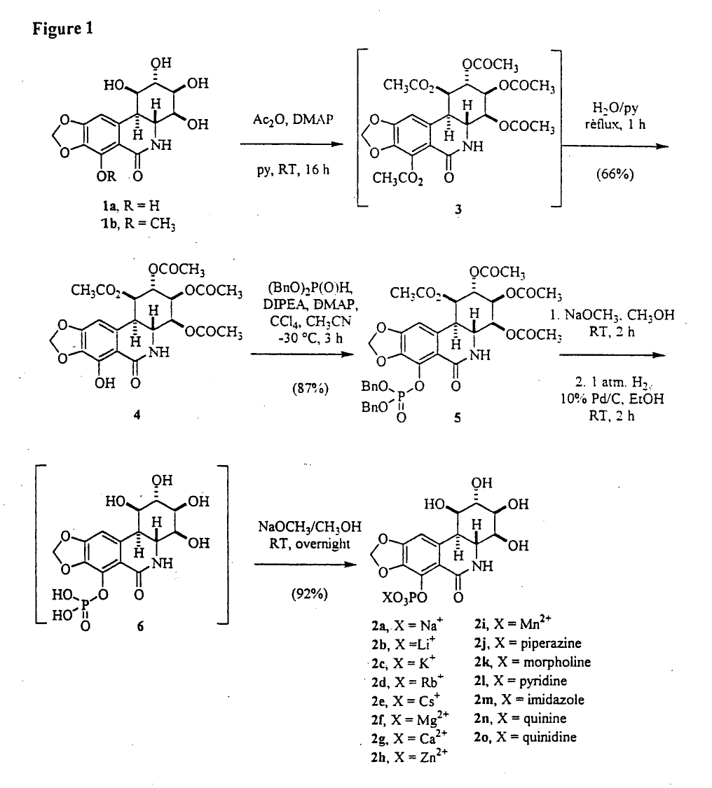

12. A method according to claim 9 wherein said phosphoric acid derivative is treated with an appropriate base so as to replace the Phosphorous atom with an ion or molecule selected from the group of structures set forth in FIG. 1 denoted as 2a-2o inclusive.
